(19) United States
(12) Patent Application Publication
(10) Pub. No.: US 2024/0316322 A1
(43) Pub. Date: Sep. 26, 2024

(54) MEDICAL DEVICE FOR THE TREATMENT OF BENIGN PROSTATIC HYPERTROPHY

(71) Applicant: Massimo MARABOTTO, Albissola Marina (IT)

(72) Inventor: Massimo MARABOTTO, Albissola Marina (IT)

(21) Appl. No.: 18/706,474

(22) PCT Filed: Sep. 20, 2022
(86) PCT No.: PCT/EP2022/076042

§ 371 Date: May 1, 2024

(30) Foreign Application Priority Data
Sep. 21, 2021 (IT) 102021000024170

(51) Int. Cl. A61M 29/02; A61M 25/00; A61M 25/10

(52) U.S. Cl. CPC A61M 29/02; A61M 25/0045; A61M 25/1011; A61M 2025/0057; A61M 2210/1096

(57) ABSTRACT

This application is directed to an at least two-way catheter comprising: a first balloon (110, 210) near the end of the catheter; a second balloon (120, 220) at a distance from the first balloon between 0 mm and 6 mm; wherein the first (140, 240) and second tube (150, 250) are coaxial, and the first tube is internal to the second tube, and wherein a drug is present on the surface of the second balloon, wherein the drug is selected from the group consisting of paclitaxel and derivatives thereof, rapamicyn and derivatives thereof, and mixtures thereof.

### FIELD OF THE INVENTION

The present invention is directed to a medical device for the treatment of benign prostatic hypertrophy, which allows a treatment with simple repeatable and outpatient procedure.

### BACKGROUND OF THE INVENTION

Benign prostatic hypertrophy (benign prostatic adenoma) is characterized by hyperplasia (ie the growth of cell number) of the parenchymal and stromal component of the gland. The hyperplasia usually occurs in the only central part of the prostate that is in direct contact with the urethra and begins with small nodules that progressively grow and increasing in volume progressively obstruct the urethral channel of outflow. Benign prostatic hypertrophy affects 5-10% of 40-year-old men with exponential growth to reach 80% of people between 70 and 80 years old.

When serenoa repens and/or alpha blocker oral therapy is not sufficient, TURP endoscopic surgical therapy (laser or diathermal loop) or open resection (open) is used. These two interventions have the serious complication of retrograde ejaculation (100% in the TURP) or total absence of it; moreover, the open intervention can also create erection problems.

Urethral catheters have long been in the state of the art. WO 98/00192 discloses an anchoring system for urethral catheters designed to prevent the catheter from positioning incorrectly both during and after insertion. The patent describes the anchoring system, does not mention its use for therapeutic modalities and release of drugs.

The use of a prostate balloon is reported in KR101487594, which describes the use of an external tube provided with a hollow shape to allow the discharge of fluid; an internal tube spaced from the outer tube; a prostate compression balloon attached to the outer tube and expanded to press the prostate; an anchor balloon to the bladder connected to the outer tube. The device is used to empty the bladder from any clots.

DE202013001998U1 discloses a catheter, which is preferably used for the treatment of functional disorders and/or diseases of the prostate and possibly of the bladder, in particular for the medicinal and/or physical treatment of the prostate, preferably in connection or after a resection transurethral and/or prostate vaporisation, in connection with benign prostatic hyperplasia or in connection with or after radiotherapy of a prostate cancer. The catheter comprises an internal tube in connection with a prostatic balloon, and an external tube in connection with a balloon to be positioned in the bladder. The drug used in the treatment is selected from an anti-phlogistic, anti-inflammatory, anti-edematous, spasmolytic, anti-cholinergic, cicatrizing, anti-septic, local anaesthetic drugs.

There is a strong need to develop a medical device that allows the treatment of benign prostatic hypertrophy by a simple and repeatable procedure.

### SUMMARY OF THE INVENTION

The present invention is directed to a medical device that includes a urethral catheter consisting of two balloons of which: one for the correct placement of the device, and the second for a mechanical action on the prostate and the simultaneous release of a drug from the surface of the balloon itself. The device allows both a pharmacological and mechanical treatment of benign prostatic hypertrophy by dosing a drug, preferably taxol or a derivative thereof, sirolimus, zotarolimus, everolimus and mixtures thereof, directly to the urethral canal and by mechanical expansion of the canal itself.

### BRIEF DESCRIPTION OF THE DRAWINGS

FIG. 1 shows the two-way system (100) in both closed and open versions. Reference number (110) corresponds to the balloon used for the correct positioning of the device. Reference number (120) corresponds to the balloon used for mechanical action on the prostate and simultaneous release of a drug from the surface of the balloon itself. Reference number (130) corresponds to the protection as a peel away introducer that covers the balloon before and during insertion and is removed before inflating it. The peel away introducer (130) prevents the drug from being dispersed along the way.

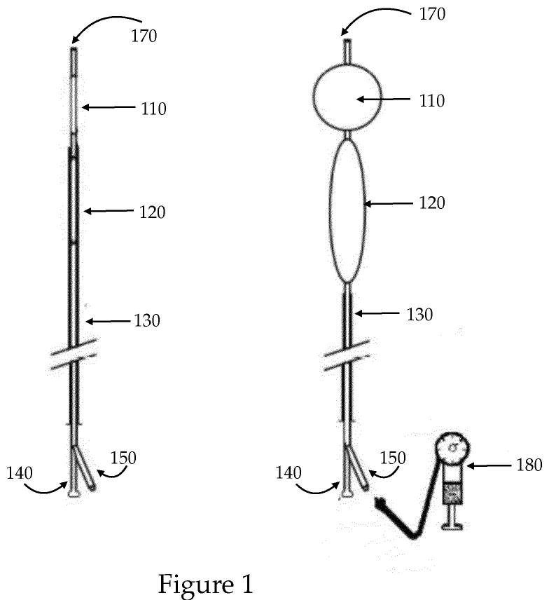

FIG. 2 shows the three-way system (200) in both closed (left side) and open (right side) configuration. The numbers (210; 220; 230) have the identical meaning as defined in FIG. 1 for numbers (110; 120; 130).

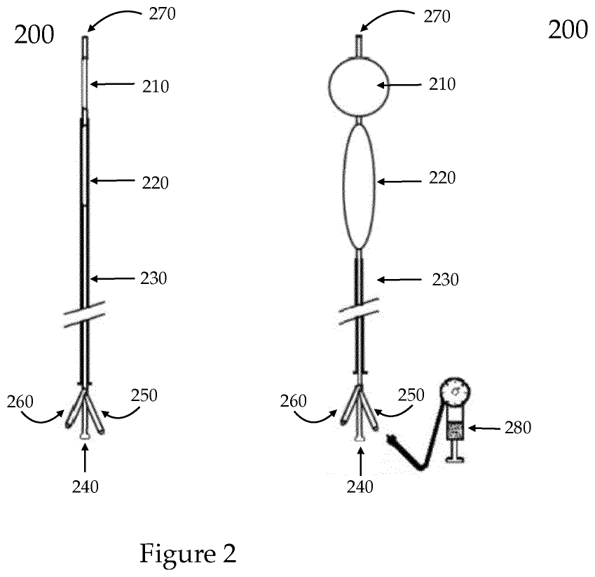

### DETAILED DESCRIPTION OF THE INVENTION

The present invention is therefore directed to a catheter comprising at least two coaxial tubes, a first tube (140; 240) for inflating a first balloon (110; 210) to be anchored in the bladder and a tube (150; 250) external to the first tube for inflating a second balloon (120; 220) necessary for dilating the urethral canal and the release of the drug. Once the catheter has been placed, the first balloon (110; 210) will be inflated in order to keep the catheter in the correct position, then it is possible to inflate the second balloon (120; 220) which dilates the urethral canal and releases the drug to it. The action of the balloon is merely mechanical, determining urethral and prostatic microfractures, the balloon is of the “semi-compliant” type, i.e. it has a predetermined size based on the amount of volume insufflated. The drug present on the surface of the balloon causes the device to act according to a double mechanical and pharmacological action by direct contact.

It is important to note that this invention makes possible for the first time a localized treatment of benign prostatic hypertrophy with chemotherapy drugs such as Paclitaxel (Taxol™) and their derivatives, and Rapamycin (Sirolimus™) and their derivatives (for example Zerolimus™ and Everolimus™). These chemotherapy drugs are currently used in arterial stenosis with excellent results and absence of side effects. The system of this invention allows a prostate treatment that is effective, repeatable, outpatient and executable in any medical office without the need for radiological or ultrasound equipment. The drugs are smeared on the surface of the second balloon, the more proximal, with methodologies already in use and widely tested and, at the expected dosage, do not present systemic side effects as demonstrated by their application in the arteries. The loss of drug during the use of the catheter according to the invention can vary from 0% to 30% depending on the type of catheter used. When using a peel away introducer, the loss is 0%. When the peel away introducer is not used the loss can be minimal or up to 30% depending on the method used to fix the drug on the balloon. The amount of drug dosed on the proximal balloon is standard and is fixed directly at the factory before marketing, as is already the case in the balloons used for arteries; the service life of the catheter is longer than three years as is the case for all sterile instruments. The application of the drug is simple and there are several tried and tested methods. The used drug is stable, does not require adjustment over time and also for this reason the duration of the catheters is at least three years. These drugs, already widely tested in vascular surgery and cardiology, associated with the dilating action of the catheter, penetrate the prostate reducing its growth and maintaining the urinary tract.

As explained above, to prevent drug dispersion during catheter placement, it is preferable to protect the balloon with a peel away introducer that covers the balloon itself during placement and prevents the medication from being dispersed along the way. Alternatively, it is possible to dose a larger amount of medication on the balloon, so that the amount of medication that is on the balloon once it reaches the correct urethral position is the amount intended to dose.

The catheter according to the invention is a catheter with at least two ways, i.e. it is composed of at least two coaxial tubes. The first tube (140; 240) allows to inflate the first balloon (110; 210) that is placed in the bladder. The second tube (150; 250), external to the first, allows to inflate the second semi-compliant balloon (120; 220) in urethral position. A three-way catheter can be used, in which the internal coaxial tube (260) can contain a guide to be placed in the bladder and then rise with the device itself.

The figures show an insufflation system with pressure gauge (180; 280) that can be used to inflate the first and second balloons. The figures also show a hole (170; 270) in tip for guide 0.35F.

In general, the diameter of the catheter will vary depending on the number of ways present, but it will in any case be between 3 mm and 10 mm. The length of the catheter is similar to that of Foley's catheters, being comprised between 35 cm and 50 cm, preferably about 40 cm.

The distance between the first balloon and the second balloon is important for the correct positioning of the second balloon and, consequently, for the release of the drug in the correct position. In the optimum solution, the distance between the first and the second balloon is between 0 mm and 6 mm.

The first balloon has dimensions similar to or equal to those of the Foley catheter balloon, with a volume, once inflated, of between 5 ml and 10 ml. The second balloon has a length between 30 mm and 50 mm, preferably between 35 mm and 45 mm and is semi-compliant. As mentioned above, the outer surface of the second balloon is sprinkled with drug. Once positioned, the second balloon is inflated to a diameter between 10 mm and 25 mm and left in place for the time needed to release the drug.

As for the amount of drug present on the balloon, it can vary in a wide range that depends among other things on the drug used. In general, the amount of drug present on the balloon is equal to or less than the maximum daily dose for the drug itself, and in general is between 0.10 mg and 3000 mg. When the drug used is paclitaxel, the amount is preferably between 1.75 mg and 300 mg, most preferably between 20 mg and 200 mg. In the case of use of sirolimus, the amount is between 0.625 mg and 3000 mg, most preferably between 10 mg and 1000 mg. For other drugs, the amount is preferably between 0.1% and 100% of the maximum daily dose, most preferably between 1% and 80%.

To fill the balloons, the corresponding way is connected to a catheter syringe, and the balloon is inflated with a predetermined amount of water. For the first balloon, an amount of water of between 5 ml and 10 ml is used, while for the second balloon it is used an amount sufficient to obtain the desired volume of the balloon.

1. A catheter (100; 200) comprising at least two ways, the catheter comprising:
a. a first balloon (110; 210) next to a first terminal of the catheter, which balloon (110; 210) is fluidically connected to a first tube (140; 240) of the catheter (100; 200);
b. a second semi-compliant balloon (120; 220) at a distance from the first balloon (110; 210) comprised between 0 mm and 6 mm and fluidically connected to a second tube (150; 250) of the catheter (100; 200);
wherein the first and second tube are coaxial, and the first tube is internal to the second tube, and wherein a drug is present on the surface of the second balloon,
characterized in that the drug is selected from the group consisting of paclitaxel and derivatives thereof, rapamicyn and derivatives thereof, and mixtures thereof.

2. The catheter according to claim 1, wherein the catheter comprises at least three ways, wherein a third internal coaxial tube (260) contains a guide.

3. The catheter according to claim 2, wherein the catheter comprises a peel away introducer (130; 230).

4. The catheter according to claim 3, wherein the drug selected from paclitaxel, Sirolimus™, Zotarolimus™, Everolimus™ and mixtures thereof.

5. The catheter according to claim 4, wherein the drug is paclitaxel and the amount present on the second balloon is between 1.75 mg and 300 mg, or the drug is sirolimus and the amount present on the second balloon is comprised between 0.625 mg and 3000 mg.

6. The catheter according to claim 1, wherein the catheter has a diameter between 4 mm and 10 mm.

7. The catheter according to claim 1, wherein the second balloon has a length of between 30 and 50 mm.

8. The catheter according to claim 1, wherein the second balloon (120; 220), when inflated, has a diameter perpendicular to the axis of the catheter comprised between 5 mm and 25 mm.

9. The catheter according to claim 1, wherein the first balloon (110; 210), when inflated, has a volume comprised between 5 ml and 20 ml.
